(19) United States
(12) Patent Application Publication
(10) Pub. No.: US 2021/0154296 A1
(43) Pub. Date: May 27, 2021

(54) VACCINE OR IMMUNOTHERAPEUTIC AGENT COMPOSITION CONTAINING PHOTOTHERMALLY TREATED CELL LYSATES AS ACTIVE INGREDIENTS

(71) Applicant: INJE UNIVERSITY INDUSTRY-ACADEMIC COOPERATION FOUNDATION, Gimhae-si, Gyeongsangnam-do (KR)

(72) Inventor: Yeon Jeong KIM, Gimhae-si, Gyeongsangnam-do (KR)

(73) Assignee: INJE UNIVERSITY INDUSTRY-ACADEMIC COOPERATION FOUNDATION, Gimhae-si, Gyeongsangnam-do (KR)

(21) Appl. No.: 16/633,168

(22) PCT Filed: Nov. 10, 2017
(86) PCT No.: PCT/KR2017/012710

§ 371 Date: Jan. 23, 2020

(30) Foreign Application Priority Data
Jul. 24, 2017 (KR) 10-2017-0093507
Oct. 18, 2017 (KR) 10-2017-0135266

(51) Int. Cl. A61K 41/00; A61K 39/00; A61P 35/00

(52) U.S. Cl. CPC A61K 41/0052; A61P 35/00; A61K 39/0011

(57) ABSTRACT

Disclosed are a vaccine composition including photothermal (PT)-treated cell lysate as an active ingredient and an immunotherapeutic composition including PT-treated cell lysate as an active ingredient, wherein the PT-treated cells exposed to ex vivo PT treatment maximize the expression of HSPs which enhance immunogenicity, thereby generating cancer-specific immune responses in vivo.

### TECHNICAL FIELD

The present invention relates to vaccine or immunotherapeutic composition comprising photothermal (PT) treated cell lysates.

### BACKGROUND ART

Cancer is generally a disease caused by abnormal maturation and excessive proliferation of cells due to mutations and damage to certain genes. The cancer cells can penetrate tissues and organs weakening their function, and can disseminate to other organs. Various types of cancers may be associated with race, sex, age, and dietary habits.

Cancer is treated by curative care, which involves removal of solidified cancer tissue or killing cancer cells directly and by palliative care; the treatment minimizes the adverse effects and delays the disease progression. In clinical practice, though significant anti-cancer effects can be achieved through surgery, chemotherapy, and radiotherapy, the three major methods of cancer treatment, these conventional strategies are insufficient to treat recurring cancer as well as cancer metastasis, which are the major causes of mortality in patients with cancer. Therefore, a variety of new strategies for treating cancers are being studied; photothermal therapy (PTT) using light is particularly attracting the attention of many researchers.

PTT is a method that helps eradicate cancer by inducing hyperthermia in the affected cells. In this therapy, cells pretreated with photoabsorbers are exposed to a laser light of a certain wavelength. The photoabsorber absorbs the laser light and generates heat energy. Photoabsorbers such as gold nanoparticles absorb light through free electrons on their surface and induce surface plasmon resonance effects. The kinetic energy generated from the light-absorbing free electrons, in turn, generates thermal energy, which eliminates the cancer cells. Owing to the higher selectivity of PTT, this treatment can be used to treat cancer with fewer adverse effects and minimal scarring as compared with conventional treatment methods such as radiotherapy or chemotherapy. The cancer inhibitory effect of PTT is also significantly higher than that of the conventional treatment methods.

On the contrary, photodynamic therapy (PDT) is a technique employed for eliminating cancer cells by using a photoabsorber, which can generate singlet oxygen upon exposure to light. Photoabsorbers accumulate in cancer cells and undergo chemical reactions using photoenergy to generate singlet oxygen and highly reactive free radicals, which can destroy cell components to induce apoptosis. Although both PTT and PDT use photoabsorbers and photoenergy, the type of photoabsorbers used and the mechanisms of action involved are different in each case. Recently, a combination of these two treatment methods has been reported to induce synergistic effects.

Despite the promising anticancer effects, lower tissue penetrating depth limits the application of PTT. Most wavelengths of light can transmit to a depth of only 2-3 mm of tissue. Although effective in the treatment of primary tumors, there are limitations to the use of light in the treatment of distant, metastasized, and recurring cancers. Therefore, there is a need for the development of more effective anti-cancer strategies.

### DISCLOSURE

### Technical Problem

It is an object of the present invention to provide a vaccine composition which can effectively inhibit cancer.

It is another object of the present invention to provide an immunotherapeutic composition that can effectively inhibit cancer.

It is another object of the present invention to provide a method of effectively increasing the immunogenicity of cells.

It is another object of the present invention to provide a method of effectively increasing the expression of heat shock proteins (HSPs) in cells.

### Technical Solution

In order to achieve the above object, the present invention provides a vaccine composition comprising PT treated cell lysate as an active ingredient.

In order to achieve the above another object, the present invention provides an immunotherapeutic composition comprising PT treated cell lysate as an active ingredient.

In order to achieve the above another object, the present invention provides a method of increasing immunogenicity of ex vivo PT treated cell lysates.

In order to achieve the above another object, the present invention provides a method of increasing HSPs of ex vivo PT treated cells.

### Advantageous Effects

The present invention maximized the expression of HSPs that increase immunogenicity of ex vivo PT treated cells thus, overcoming the limitations of PTT, such as a low exposure efficiency and tissue damage during laser irradiation in vivo. Since specific immune responses can be generated more efficiently than conventional cancer vaccines, the lysates obtained from PT treated cells can be used as vaccines or in immunotherapeutic compositions.

### DESCRIPTION OF DRAWINGS

FIG. 1 shows a result of the temperature increase and the cell viability change according to the ex vivo PT treatment and heating according to an embodiment of the present invention.

FIG. 2 shows the expression levels of HSP70 in ex vivo PT treated cells and heated cells according to an embodiment of the present invention.

FIG. 3 shows the expression levels of HSP27 (left) and HSP90 (right) in cancer cells after ex vivo PT treatment according to an embodiment of the present invention (100 μg/ml-4 W/cm², bold line; 20 μg/ml-4 W/cm², solid line; 4 μg/ml-4 W/cm², sparse dashed line; water bath heating, dense dashed line).

FIG. 4 shows the cancer antigen specific target lysis in vivo induced by the vaccination of ex vivo PT-treated cancer cell lysates-pulsed DCs, according to an embodiment of the present invention.

FIG. 5 shows in vivo anticancer effect of the DC vaccine using ex vivo PT-treated CT-26-HER2/neu cell lysates according to an embodiment of the present invention.

### BEST MODE

Hereinafter, the present invention will be described in more detail.

The present invention provides a vaccine composition comprising PT treated cell lysate as an active ingredient.

In this case, the PT treatment may be performed by using at least any one photoabsorber selected from the group consisting of indocyanine green (ICG), gold nanorod, gold nanosphere and carbon chitosan covered with CuS, and any material that can be used as the photoabsorber may be used, but it is not limited thereto.

The PT treatment may be performed by irradiating a laser of any one of wavelength band selected from the group consisting of 360-430 nm, 480-680 nm, 630-670 nm, 700-2500 nm and 780-850 nm, but it is not limited thereto.

In addition, the PT treatment may be performed by irradiating a laser at an intensity of 0.5 W/cm² to 20 W/cm², preferably 2 W/cm² to 4 W/cm² and more preferably 4 W/cm². In conventional PTT, since the laser is used directly on patients, there is a limit to the intensity of the laser, which can be used to reduce adverse effects. Since the skin penetration efficiency of laser is extremely low, the intensity of the laser to which cancer cells are exposed through PTT is also limited. In contrast, according to the present invention, PT treatment is used to maximize the expression of HSPs. Thus, a laser of high intensity can be directly irradiated onto the cancer cells ex vivo, thereby improving immunogenicity and maximizing the expression of HSPs, which in turn contribute to immunogenicity.

In addition, the PT treatment may be performed by increasing the temperature of cancer cells from 35° C. to 100° C., preferably between 40° C. to 65° C. This temperature range can be used to maximize the expression of the HSPs and the immunogenicity, which is desirable.

In one embodiment of the present invention, the cell may be any one selected from the group consisting of cancer cells, pathogen-infected cells, antigen-loaded immune cells and immunogenic cells.

In addition, the PT treatment is preferably performed ex vivo.

The vaccine composition of the present invention may comprise a pharmaceutically acceptable carrier. It means any component suitable for delivering an antigenic substance to a site in vivo, for example, water, saline, phosphate buffered saline, Ringer's solution, dextrose solution, serum-containing solution, Hans' solution, other water soluble physiological equilibrium solutions, oils, esters and glycols, and the like, but it is not limited thereto.

The carrier may comprise suitable auxiliaries and preservatives to enhance chemical stability and isotonicity, and may include stabilizers such as trehalose, glycine, sorbitol, lactose or monosodium glutamate (MSG) and thus can protect the vaccine composition against temperature changes or lyophilization. The vaccine composition of the present invention may comprise a suspension liquid, such as sterile water or saline (preferably buffered saline).

The vaccine composition of the present invention may contain any adjuvant in an amount sufficient to enhance the immune response to the immunogen. Suitable adjuvants are described in Takahashi et al. (1990) Nature 344: 873-875, for example, aluminum salts (aluminum phosphate or aluminum hydroxide), squalene mixtures (SAF-1), muramyl peptides, saponin derivatives, mycobacterial cell wall products, monophosphoryl lipid A, mycolic acid derivatives, nonionic block copolymer surfactants, Quil A, cholera toxin B subunits, polyphosphazenes and derivatives, and immunostimulatory complexes (ISCOMs), it is not limited thereto.

Like all other vaccine compositions, the immunologically effective amount of an immunogen should be determined empirically, factors that can be considered in this case include immunogenicity, route of administration and the number of times of vaccine administration.

According to the present invention, cell lysates, which act as antigens in the anti-cancer vaccine may be present at various concentrations, but are typically included at concentrations necessary for the induction of appropriate levels of antibodies in vivo.

The vaccine composition can be administered via the conventional method such as intravenous, intraarterial, intraperitoneal, intramuscular, intraosseous, transdermal, nasal, inhalation, topical, rectal, oral, intraocular, subcutaneous or intradermal routes.

In addition, the present invention provides an immunotherapeutic composition comprising a PT treated cell lysate as an active ingredient.

In this case, the PT treatment may be performed by using at least any one photoabsorber selected from the group consisting of ICG, gold nanorod, gold nanosphere and carbon chitosan covered with CuS, and any material that can be used as the photoabsorber may be used, but it is not limited thereto.

The PT treatment may be performed by irradiating a laser of any one of wavelength band selected from the group consisting of 360-430 nm, 480-680 nm, 630-670 nm, 700-2500 nm and 780-850 nm, but it is not limited thereto.

In addition, the PT treatment may be performed by irradiating a laser at an intensity of 0.5 W/cm² to 20 W/cm², preferably 2 W/cm² to 4 W/cm² and more preferably 4 W/cm². In conventional PTT, since the laser is used directly on patients, there is a limit to the intensity of the laser, which can be used to reduce adverse effects. Since the skin penetration efficiency of laser is extremely low, the intensity of the laser to which cancer cells are exposed through PTT is also limited. In contrast, according to the present invention, PT treatment is used to maximize the expression of HSPs. Thus, a laser of high intensity can be directly irradiated onto the cancer cells ex vivo, thereby improving immunogenicity and maximizing the expression of HSPs, which in turn contribute to immunogenicity.

In addition, the PT treatment may be performed by increasing the temperature of cancer cells from 35° C. to 100° C., preferably between 40° C. to 65° C. This temperature range can be used to maximize the expression of the HSPs and the immunogenicity, which is desirable.

In one embodiment of the present invention, the cell may be any one selected from the group consisting of cancer cells, pathogen-infected cells, antigen-loaded immune cells and immunogenic cells.

In addition, the PT treatment is preferably performed ex vivo.

In one embodiment of the present invention, the immunotherapeutic composition can be used as any one formulation selected from the group consisting of injections, granules, powders, tablets, pills, capsules, suppositories, gels, suspensions, emulsions, drops and solutions according to conventional methods.

In another embodiment of the invention, the immunotherapeutic composition may comprise at least one additive selected from the group consisting of carriers, excipients, disintegrants, sweeteners, coating agents, swelling agents, slip modifiers, flavors, antioxidants, buffers, bacteristats, diluents, dispersants, surfactants, binders and lubricants, which is suitably used for the preparation of a pharmaceutical composition.

Examples of the carrier, excipient and diluent include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oil. Solid formulations for oral administration include tablets, pills, powders, granules, capsules, etc., and such solid formulations may contain at least one excipient such as starch, calcium carbonate, sucrose or lactose, gelatin and the like in addition to the composition. Furthermore, in addition to simple excipients, lubricants such as magnesium stearate and talc are also used. Examples of the liquid formulations for oral administration include suspensions, solutions, emulsions, syrups and the like, and various excipients such as wetting agents, sweeteners, fragrances, preservatives and the like may be included in addition to water and liquid paraffin which are commonly used as simple diluents. Formulations for parenteral administration include sterile aqueous solutions, non-aqueous solutions, suspensions, emulsions, freeze-dried preparations, suppositories and the like. Examples of the non-aqueous solution and the suspension include propylene glycol, polyethylene glycol, vegetable oil such as olive oil, injectable ester such as ethyl oleate, and the like. As the base of the suppository, witepsol, macrogol, tween 61, cacao butter, laurinum, glycerogelatin and the like can be used.

According to one embodiment of the invention the immunotherapeutic composition can be administered via the conventional method such as intravenous, intraarterial, intraperitoneal, intramuscular, intraosseous, transdermal, nasal, inhalation, topical, rectal, oral, intraocular, subcutaneous or intradermal routes.

In addition, the present invention provides a method of increasing immunogenicity of cells included in ex vivo PT treated cell lysates.

The present invention also provides a method of increasing expression of HSPs of cells included in ex vivo PT treated cell lysates.

In this case, the HSP may be selected from the group consisting of HSP10, HSP27, HSP47, HSP56, HSP60, HSP70, HSP90 and HSP110, but it is not limited thereto.

In this case, the PT treatment may be performed by using at least any one photoabsorber selected from the group consisting of ICG, gold nanorod, gold nanosphere and carbon chitosan covered with CuS, and any material that can be used as the photoabsorber may be used, but it is not limited thereto.

The PT treatment may be performed by irradiating a laser of any one of wavelength band selected from the group consisting of 360-430 nm, 480-680 nm, 630-670 nm, 700-2500 nm and 780-850 nm, but it is not limited thereto.

In addition, the PT treatment may be performed by irradiating a laser at an intensity of 0.5 W/cm² to 20 W/cm², preferably 2 W/cm² to 4 W/cm² and more preferably 4 W/cm². In conventional PTT, since the laser is used directly on patients, there is a limit to the intensity of the laser, which can be used to reduce adverse effects. Since the skin penetration efficiency of laser is extremely low, the intensity of the laser to which cancer cells are exposed through PTT is also limited. In contrast, according to the present invention, PT treatment is used to maximize the expression of HSPs. Thus, a laser of high intensity can be directly irradiated onto the cancer cells ex vivo, thereby improving immunogenicity and maximizing the expression of HSPs, which in turn contribute to immunogenicity.

In addition, the PT treatment may be performed by increasing the temperature of cancer cells from 35° C. to 100° C., preferably between 40° C. to 65° C. This temperature range can be used to maximize the expression of the HSPs and the immunogenicity, which is desirable.

In one embodiment of the present invention, the cell may be any one selected from the group consisting of cancer cells, pathogen-infected cells, antigen-loaded immune cells and immunogenic cells.

Hereinafter, the present invention will be described in detail with reference to the following examples. The examples are only for describing the present invention in more detail and it is obvious to those skilled in the art that that the scope of the present invention is not limited by these examples embodiments in accordance with the gist of the present invention.

### <Example 1> Changes in Temperature and Cell Viability of PT Treated Cells Using ICG

Since PTT is a treatment method that involves the use of light, the type of light used is important. The most commonly used type is a laser at a wavelength of 808 nm, which can induce hyperthermia efficiently. In the present invention, ICG, which has been proved safe for clinical use as a photoabsorber is employed. First, it was confirmed whether ICG was effectively taken up into CT-26-HER2/neu, a cancer cell line, and that hyperthermia could be generated after laser irradiation.

Specifically, ICG (MPbio) was taken up into CT-26-HER2/neu cell line to measure the tissue temperature, which corresponded to the efficiency of PT treatment. CT-26-HER2/neu cells at a concentration of 5×10⁵ were pre-seeded in 96 well plates for 24 h. ICG stock (10 mg/mL) were prepared at concentrations of 4 μg/mL, 20 μg/mL, and 100 μg/mL each. Tumor cells were incubated in the presence of ICG for 90 min. Thereafter, the cells were washed with warm PBS and resuspended in 50 μL of fresh DMEM. The cells were then irradiated with lasers of intensity 1 W/cm², 2 W/cm², and 4 W/cm² respectively, for 100 s to 500 s and were allowed a recovery time of 1 h. It is important to irradiate all samples with laser at a constant distance from the cells.

The same number of cells as that used in the PT treatment were seeded in 96-well plates for 24 h; they were resuspended in a small amount of medium, the following day, and then heated in a water bath, allowing recovery for 1 h.

The changes in the temperature were measured immediately after laser irradiation or water bath heating and the cell viability was measured after 1 h of recovery time.

Cells exposed to PT treatment or water bath heating were placed in 96-well plates, and WST solution (EZ-Cytox, Dogen) was added. The cell viability of the cancer cells was confirmed by measuring their O.D value at 450 nm using the XFlour software when the color of the reagent changed to the appropriate levels.

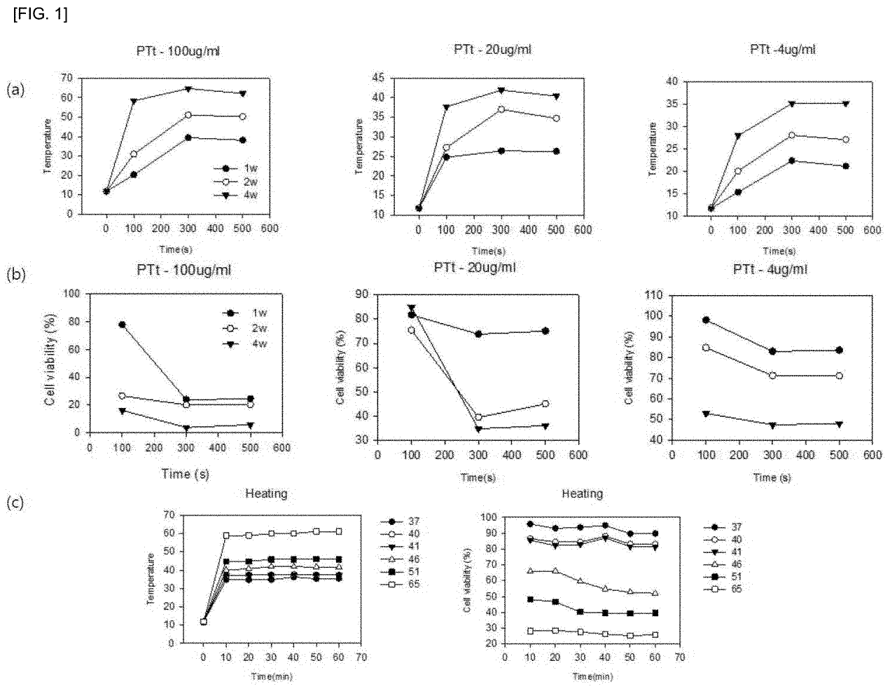

As described above, we measured the increase in the temperature of cancer cell lines [FIG. 1(a)] and the decrease in cell viability [FIG. 1(b)] at each concentration of ICG used. After exposure to 100 μg/mL of ICG and laser of wavelength 808 nm at an intensity of 4 W/cm², the temperature of cells increased to 65° C. and the cell viability was less than 10%. When the cancer cells were heated by using the conventional technique of incubation in warm water at 65° C. for 1 h, their temperature increased to about 60° C. while their viability decreased [FIG. 1(c)].

### <Example 2> Induction of HSP Expression

A strategy to increase the immunogenicity of cancer cells through heating in a water bath had been used previously. Heat treatment increases the expression of HSPs in cancer cells. HSPs, a type of chaperones, are known not only to provide protection against thermal stress, but also enhance the immunogenicity of cancer antigens by binding to them. Specifically, when the tumor is taken up by dendritic cells (DC), the antigens present in the tumor cells are processed inside the DCs, and are bound to HSPs. When used as a cancer vaccine, HSPs contribute to the activation of a stronger antigen-specific immune response. HSP70 has been reported to play a major role in increasing immunogenicity against cancer.

CT-26-HER2/neu cells and ICG were co-cultured in 96 well plates for 24 h prior to PTT and irradiated with the laser for 300 s at each of the intensities (W/cm²) as described in Example 1. Fix/Perm staining was then performed after allowing 1 h for HSP recovery using the Fix/Perm kit in accordance with the protocol of the product. This was followed by staining with PE-labeled anti-HSP70 antibody (Santacruz) for 1 h and analyzing by flow cytometry (CELLQuest software).

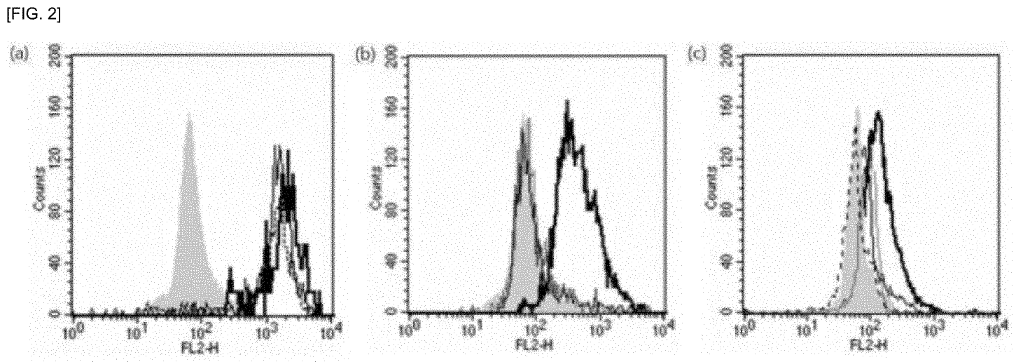

As the obtained results suggest, PT treatment with ICG at a concentration of 100 μg/mL led to a significant increase in HSP70 expression irrespective of the laser intensity used [FIG. 2(a)]. Even at a concentration of 20 μg/mL ICG, low levels of HSPs were induced by laser at intensities of 1 W/cm² and 2 W/cm², but laser at an intensity of 4 W/cm² induced relatively higher levels of HSPs than the cells-only or heat-treated groups [FIG. 2(b)]. The conventional technique of heating using a water bath, induced optimal HSP70 expression at 41° C., but the levels were relatively low compared to those after PT treatment. Increased levels of HSP70 were not induced even when the cells were heated to temperatures higher than 41° C. [FIG. 2(c)].

### <Example 3> Induction of HSP27 and HSP90 Expression

To determine whether the expression of other HSPs can contribute to immunogenicity induced by ex vivo PT treatment in addition to HSP70, the expression levels of HSP27 and HSP90 were measured in cancer cells after PT treatment. More specifically, CT-26-HER2/neu cells were seeded in 96-well plates 24 h before PT treatment, and then treated with ICG and irradiated with lasers at each of the intensities and optimal time in the same manner as described in Example 2. Fix/Perm staining was performed after allowing a recovery time of 1 h after PT treatment using a Fix/Perm kit according to the product's protocol. Staining was then performed using FITC-labeled anti-HSP27 (ENZO) and PE-labeled anti-HSP90 antibodies (ENZO) for 1 h and analyzed using flow cytometry.

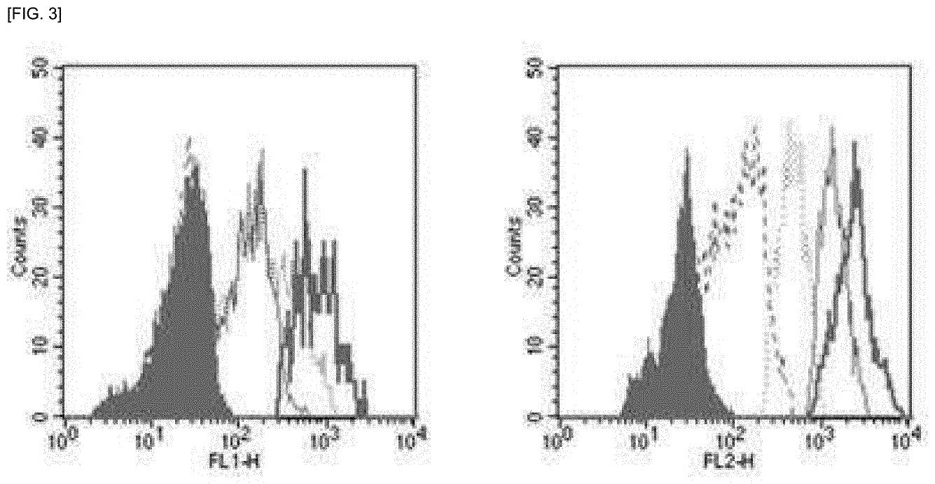

The results, as shown in FIG. 3, indicate that both HSP27 (left of FIG. 3) and HSP90 (right of FIG. 3) levels increased in proportion to the ICG concentration and the laser intensity used, similar to the increase in the levels of HSP70 observed previously. Therefore, from these results, it can be concluded that ex vivo PT treatment may contribute to improving immunogenicity by efficiently increasing the expression levels of various HSPs.

### <Example 4> Analysis of Immunogenicity of PT Treated Cell Lysates

(1) Preparation of DC Vaccine Comprising of PT-Treated Cancer Cell Lysates

In order to examine whether the immunogenicity of PT-treated cancer cells was actually enhanced, a DC vaccine using cancer cell lysates was prepared and administered to mice.

Specifically, DCs were obtained by differentiation of bone marrow cells after 6 days of culture in a medium containing GM-CSF. In the same manner as described above, CT-26-HER2/neu cells and ICG were co-cultured and PT treatment was performed. After allowing 1 h of recovery, cells were collected in Eppendorf tubes and subjected to 5 cycles of freezing in liquid nitrogen and thawing in a water bath at 37° C. for 15 min each. The supernatant was then collected by centrifugation centrifugation (12000 rpm, 10 minutes, 4° C.) and was pulsed into DCs for 24 h as described above. For the control DC vaccine, CT-26-HER2/neu cells were seeded with the same number of cells as were heated in the water bath. Thereafter, five cycles of freezing and thawing were performed in the same manner as described above, and the supernatants were taken and pulsed into DCs for 24 h.

(2) Analysis of Immunogenicity of DC Vaccine

Naïve BALB/c mice (Orient) were immunized with the DC vaccine prepared by the methods described above by subcutaneous injection. Two weeks later, the mice were sacrificed and the spleen was removed to obtain splenocytes. The splenocytes were then divided into two batches and used as peptide non-pulsed and pulsed groups. The peptide pulsed group was treated with p63 peptide (CTL epitope of Her-2/neu cancer antigen) for 90 min. CFSE labeling was performed after 15 min. The peptide pulsed group was labeled at a high dose of CFSE and the non-pulsed group was labeled at a low dose of CFSE. Each group of labeled cells (1×10⁷/mice) was then injected intravenously into mice preimmunized for two weeks. After 24 h, the mice were sacrificed to isolate splenocytes. Splenocytes were analyzed for HER2 peptide specific CTL activity using FACS.

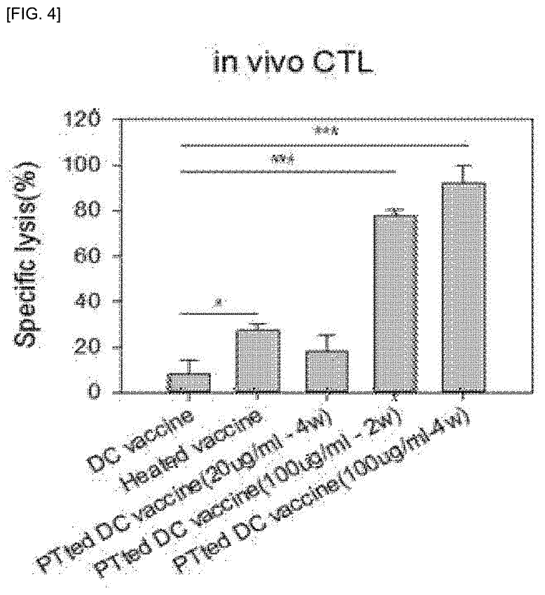

The results, as shown in FIG. 4, indicate that more than 90% antigen-specific target cell lysis was induced in mice immunized with DC vaccines pulsed with cell lysates from cancer cells exposed to laser of intensity 4 W/cm² after treatment with 100 μg/mL ICG. Eighty percent of antigen specific target cell lysis was observed in mice immunized with PT-treated DC vaccine (100 μg/mL ICG, laser at intensity of 2 W/cm²). In contrast, in mice vaccinated with the heated DC vaccine, only 30% of the target cells were removed in an antigen specific manner. Thus, immunization with DC vaccines using PT-treated cancer cell lysates efficiently induced cytotoxic T-cell responses, and their levels were significantly higher than those observed using the conventional technique of DC vaccines that uses heated cancer cell lysates.

### <Example 5> Induction of In Vivo Anticancer Effect

Mice were injected subcutaneously with CT-26-HER2/neu cells at a concentration of 3×10⁵ cells/mouse. After 24 h, the immunization with DC vaccine was performed under the conditions as described previously. The anticancer effects induced by immunization with the vaccine were analyzed.

More specifically, mice were inoculated with CT-26-HER2/neu cells by subcutaneous injection at a concentration of 3×10⁵ cells/mouse. After 24 h, DC vaccines pulsed with cancer cell lysates and heated cancer cell lysates prepared by the method described in Example 4 were subcutaneously injected into the mice. Thereafter, the tumor growth levels in the mice of each group were analyzed at two-day intervals and compared with each other.

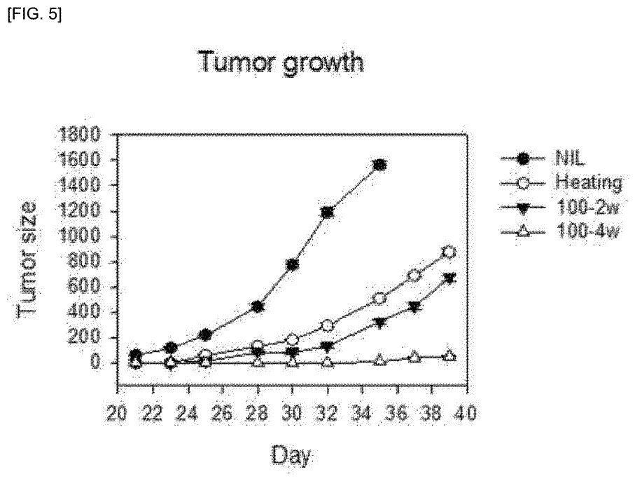

The results, as shown in FIG. 5 indicate that tumor growth was inhibited significantly in the group immunized with DC vaccine which was pulsed with cell lysates from cancer cells treated with 100 μg/mL of ICG and irradiated with laser at 4 W/cm², compared with other groups. PT-treated DC vaccine prepared after incubation with 100 μg/mL ICG and exposed to laser at intensity of 2 W/cm² had the highest anticancer efficacy in the following order, and heated DC vaccine had the lowest anticancer efficacy among them. As a result, we can assume that immunization with vaccine prepared according to the present invention, results in the induction of better anti-cancer effects, compared with immunization with vaccine prepared using the conventional technology.

While the present invention has been particularly described with reference to specific embodiments thereof, it is apparent that this specific description is only a preferred embodiment and that the scope of the present invention is not limited thereby to those skilled in the art. That is, the practical scope of the present invention is defined by the appended claims and their equivalents.

1. A method of inducing immune response, comprising:
preparing a vaccine composition comprising PT treated cell lysate as an active ingredient; and
administering the vaccine composition to a subject.

2. The method of claim 1, wherein PT treatment is performed by using at least any one photoabsorber selected from the group consisting of indocyanine green, gold nanorod, gold nanosphere and carbon chitosan covered with CuS.

3. The method of claim 1, wherein PT treatment is performed by irradiating a laser of any one of wavelength band selected from the group consisting of 360-430 nm, 480-680 nm, 630-670 nm, 700-2500 nm and 780-850 nm.

4. The method of claim 1, wherein PT treatment is performed by irradiation with a laser at an intensity of 0.5 W/cm2 to 20 W/cm2.

5. The method of claim 1, wherein the PT treatment is performed by increasing temperature of the cell of 35° C. to 100° C.

6. The method of claim 1, wherein the cell lysate is from any one selected from the group consisting of cancer cell, pathogen-infected cell, antigen-loaded immune cell and immunogenic cell.

7. The method of claim 1, wherein PT treatment is performed ex vivo.

8. A method of inducing anti-cancer effect, comprising:
preparing an immunotherapeutic composition comprising PT treated cell lysate as an active ingredient; and
administering the immunotherapeutic composition to a subject.

9. The method of claim 8, wherein PT treatment is performed by using at least any photoabsorber selected from the group consisting of indocyanine green, gold nanorod, gold nanosphere and carbon chitosan covered with CuS.

10. The method of claim 8, wherein the PT treatment is performed by irradiating a laser of any one of wavelength band selected from the group consisting of 360-430 nm, 480-680 nm, 630-670 nm, 700-2500 nm and 780-850 nm.

11. The method of claim 8, wherein PT treatment is performed by irradiating a laser at an intensity of 0.5 W/cm2 to 20 W/cm2.

12. The method of claim 8, wherein PT treatment is performed by increasing temperature of the cell of 35° C. to 100° C.

13. The method of claim 8, wherein the cell is any one selected from the group consisting of cancer cell, pathogen-infected cell, antigen-loaded immune cell and immunogenic cell.

14. The method of claim 8, wherein PT treatment is performed ex vivo.

15. A method of increasing immunogenicity of cells included in ex vivo PT treated cell lysates.

16-17. (canceled)

18. The method of claim 1, wherein PT treatment enhances immunogenicity of cell lysate.

19. The method of claim 8, wherein PT treatment enhances immunogenicity of cell lysate.
